(19) United States
(12) Patent Application Publication
(10) Pub. No.: US 2022/0218008 A1
(43) Pub. Date: Jul. 14, 2022

(54) TASTE MODULATING COMPOUNDS AND METHODS OF IMPROVING THE QUALITY OF FOODS AND BEVERAGES

(71) Applicant: Ohio State Innovation Foundation, Columbus, OH (US)

(72) Inventor: Devin PETERSON, Columbus, OH (US)

(21) Appl. No.: 17/612,779

(22) PCT Filed: May 21, 2020
(86) PCT No.: PCT/US2020/034016

§ 371 Date: Nov. 19, 2021

Related U.S. Application Data
(60) Provisional application No. 62/850,906, filed on May 21, 2019.

(51) Int. Cl. A23L 27/00; A23L 27/20; A23L 2/56; C12C 5/02; C12G 1/00; A21D 2/14; A23C 9/13; C12G 3/06; A61K 47/14

(52) U.S. Cl. CPC A23L 27/84; A23L 27/86; A23L 27/204; A23L 2/56; C12C 5/026; A23V 2002/00; A21D 2/14; A23C 9/1307; C12G 3/06; A61K 47/14; C12G 1/00

(57) ABSTRACT

Disclosed herein are small molecule compounds that can be used to modulate flavors in foods, beverages, and other articles intended for delivery to the oral cavity. The compounds include cinnamoyl and caffeic esters as disclosed herein. Suitable foods and beverages include coffee, beers.

### CROSS-REFERENCE TO RELATED APPLICATION

This application claims the benefit of U.S. Provisional Application 62/850,906, filed on May 21, 2019, the contents of which are hereby incorporated in its entirety.

### FIELD OF THE INVENTION

The invention relates to flavor modulating compounds and their use as an additive for food and drink. By blocking/modulating bitter and/or sour flavors, the amount of added sugars and/or salts can be reduced.

### BACKGROUND

There are five primary tastes perceived by the human tongue: salt, sour, sweet, bitter, and umami (i.e., savory). Many people consider the bitter sensation to be unpleasant, and it is speculated that the ability to sense bitterness evolved as an avoidance mechanism against toxic plants and animals. Nevertheless, many foods with high nutritional value, for instance cruciferous vegetables and cranberries, also have bitter flavors. Similarly, foods that are overly sour are not well tolerated by a majority of consumers as well. These foods are often prepared with high levels of fats, sugars, and/or salts in order to mask the bitterness. Although these additives increase the palatability of the nutritious foods, excess consumption of fat, sugar, and salt is considered unhealthy. As an alternative, bitter blocking compounds having been developed as an additive for foods and vegetables. However, different foods have different distributions of bitter compounds, and many additives only block a subset of bitter flavors.

Compounds with sour modulating behavior are even less well known in the literature. In many products such as yogurt or juice, excessive sourness can occur and negatively impact the product quality. Currently the citrus industry in Florida (also California) is in a critical situation with huanglongbing (HLB) or citrus greening disease. Juice produced from HLB infected fruit is low in quality, with increased bitterness and sourness, as well as a poor aroma. Earlier research showed that HLB-induced off-flavor was not detectable in juice made with up to 25% symptomatic fruit in healthy juice. However, increasing the concentration of symptomatic juice presents a detectable and recognizable off flavor. Moreover, in many regions it is increasingly difficult to find fruit not showing HLB symptoms. Although elimination of the causative organism of citrus greening would be the ideal solution, there remains a need for methods of improving the flavor quality of HLB-induced off flavors.

There remains a need for novel compounds that block the bitter and/or sour sensations in foods and beverages. The remains a need for compounds that modulate the aromas of foods and beverages. There remains a need for palatable foodstuffs with reduced sugar and salt additives.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The term “alkyl” as used herein is a branched or unbranched hydrocarbon group such as methyl, ethyl, n-propyl, isopropyl, n-butyl, isobutyl, t-butyl, pentyl, hexyl, heptyl, octyl, nonyl, decyl, dodecyl, and the like. The alkyl group can also be substituted or unsubstituted. Unless stated otherwise, the term “alkyl” contemplates both substituted and unsubstituted alkyl groups. The alkyl group can be substituted with one or more groups including, but not limited to, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. An alkyl group which contains no double or triple carbon-carbon bonds is designated a saturated alkyl group, whereas an alkyl group having one or more such bonds is designated an unsaturated alkyl group. Unsaturated alkyl groups having a double bond can be designated alkenyl groups, and unsaturated alkyl groups having a triple bond can be designated alkynyl groups. Unless specified to the contrary, the term alkyl embraces both saturated and unsaturated groups.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is replaced with a heteroatom such as, but not limited to, nitrogen, oxygen, sulfur, selenium or phosphorus. The cycloalkyl group and heterocycloalkyl group can be substituted or unsubstituted. Unless stated otherwise, the terms “cycloalkyl” and “heterocycloalkyl” contemplate both substituted and unsubstituted cyloalkyl and heterocycloalkyl groups. The cycloalkyl group and heterocycloalkyl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. A cycloalkyl group which contains no double or triple carbon-carbon bonds is designated a saturated cycloalkyl group, whereas an cycloalkyl group having one or more such bonds (yet is still not aromatic) is designated an unsaturated cycloalkyl group. Unless specified to the contrary, the term cycloalkyl embraces both saturated and unsaturated, non-aromatic, ring systems.

The term “aryl” as used herein is an aromatic ring composed of carbon atoms. Examples of aryl groups include, but are not limited to, phenyl and naphthyl, etc. The term “heteroaryl” is an aryl group as defined above where at least one of the carbon atoms of the ring is replaced with a heteroatom such as, but not limited to, nitrogen, oxygen, sulfur, selenium or phosphorus. The aryl group and heteroaryl group can be substituted or unsubstituted. Unless stated otherwise, the terms “aryl” and “heteroaryl” contemplate both substituted and unsubstituted aryl and heteroaryl groups. The aryl group and heteroaryl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol.

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyL cirrnolinyl, decahydroquinolinyl, 2H,6H˜ 1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

The terms “alkoxy,” “cycloalkoxy,” “heterocycloalkoxy,” “cycloalkoxy,” “aryloxy,” and “heteroaryloxy” have the aforementioned meanings for alkyl, cycloalkyl, heterocycloalkyl, aryl and heteroaryl, further providing said group is connected via an oxygen atom.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Unless specifically stated, a substituent that is said to be “substituted” is meant that the substituent can be substituted with one or more of the following: alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. In a specific example, groups that are said to be substituted are substituted with a protic group, which is a group that can be protonated or deprotonated, depending on the pH.

Acceptable salts are salts that retain the desired flavor modulating activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, naphthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, and all possible geometric isomers.

Disclosed herein are flavor modulating compounds having the formula:

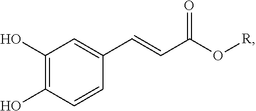

wherein R is C₁₋₈alkyl, C₂₋₈alkenyl, C₂₋₈alkynyl, aryl, C₁₋₈heteroaryl, C₃₋₈cycloalkyl, or C₁₋₈heterocyclyl. In certain preferred embodiments, R can be a C₃₋₈cycloalkyl or aryl group having the formula:

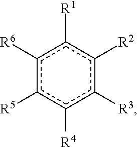

wherein at least one of R¹, R², R³, R⁴, R⁵, or R⁶ represents a bond to a cinnamoyl group, [symbol] independently represents a single or double bond, as permitted by valence, and the remaining five groups are selected from hydrogen, OH, C(O)OH, O—C₁₋₈alkyl, C(O)O—C₁₋₈alkyl, C₁₋₈alkyl, CH₂OH, and CH₂OC₁₋₈alkyl.

In some embodiments, R¹ and R² are bonds to a cinnamoyl group and R³, R⁴, R⁵, and R⁶ are as defined above. In some embodiments, R¹ and R³ are bonds to a cinnamoyl group and R², R⁴, R⁵, and R⁶ are as defined above. In some embodiments, R¹ and R⁴ are bonds to a cinnamoyl group and R², R³, R⁵, and R⁶ are as defined above.

In some embodiments, R can be a cyclitol group. As used herein, a cyclitol group is a cycloalkyl group, preferably a C₆ cycloalkyl group having at least two hydroxyl groups. For instance, R can be a bornesitol group connected to the cinnamoyl group at the 1, 2, 3, 4, or 5 hydroxyl position; R can be a conduritol group connected to the cinnamoyl group at the 1, 2, 3, or 4 hydroxyl position; R can be a inositol group connected to the cinnamoyl group at the 1, 2, 3, 4, 5, or 6 hydroxyl position; R can be a pinitol group connected to the cinnamoyl group at the 1, 2, 3, 4, or 5 hydroxyl position; R can be a pinpollitol group connected to the cinnamoyl group at the 1, 2, 4, or 5 hydroxyl position; R can be a quebrachitol group connected to the cinnamoyl group at the 1, 2, 3, 4, or 5 hydroxyl position; R can be a quinic acid group connected to the cinnamoyl group at the 1, 3, 4, or 5 hydroxyl position; R can be a shikimic acid group connected to the cinnamoyl group at the 3, 4, or 5 hydroxyl position; R can be a valienol group connected to the cinnamoyl group at the 1, 2, 3, or 4 hydroxyl position; or R can be a viscumitol group connected to the cinnamoyl group at the 1, 2, 3, 4, or 5 hydroxyl position. When R is quinic acid or shikimic acid, the carboxylic acid group may be further esterified, such as with a C₁₋₈alkyl group.

In some embodiments, the flavor modulating compounds can have the structure:

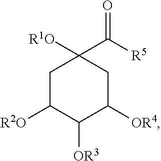

wherein at least one of R¹, R², R³, or R⁴ is a caffeic acid derivative having the formula:

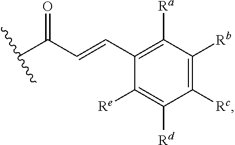

wherein R^{a}, R^{b}, R^{c}, R^{d}, and R^{e} are independently selected from F, Cl, Br, I, nitro, R, OR, N(R)₂, SO₂R, SO₂N(R)₂, C(O)R; C(O)OR, OC(O)R; C(O)N(R)₂, N(R)C(O)R, OC(O)N(R^{1b′})₂, N(R)C(O)N(R)₂, wherein R is in each case independently selected from hydrogen, C₁₋₈alkyl, C₂₋₈ alkenyl, C₂₋₈alkynyl, aryl, C₁₋₈heteroaryl, C₃₋₈cycloalkyl, or C₁₋₈heterocyclyl;

the remaining R¹, R², R³, and R⁴ are independently selected from hydrogen, C(O)R; C(O)OR, and C(O)N(R)₂, wherein R is in each case independently selected from hydrogen, C₁₋₈ alkyl, C₂₋₈alkenyl, C₂₋₈alkynyl, aryl, C₁₋₈heteroaryl, C₃₋₈cycloalkyl, or C₁₋₈heterocyclyl;

R⁵ is selected from OR or NR₂, wherein R is independently selected from hydrogen, C₁₋₈ alkyl, C₂₋₈alkenyl, C₂₋₈alkynyl, aryl, C₁₋₈heteroaryl, C₃₋₈cycloalkyl, or C₁₋₈heterocyclyl. In cases where R⁵ is OH, acceptable salts as defined above are also included. In some embodiments, R⁵ may form a bond with any of R¹, R², R³, or R⁴.

In some instances, it is preferred that R⁵ is a group having the formula —O—C₁₋₆alkyl. Exemplary C₁₋₆alkyl include methyl, ethyl, n-propyl, isopropyl, n-butyl, isobutyl, sec-butyl, and t-butyl. It can also be preferred that each of the R¹, R², R³, or R⁴ that is not the caffeic acid derivative will be hydrogen. For instance, R⁴ can be the caffeic acid derivative and each of R¹, R², R³, and R⁵ are hydrogen. In other instances, R³ can be the caffeic acid derivative and each of R¹, R², R⁴, and R⁵ are hydrogen. In further embodiments, R² can be the caffeic acid derivative and each of R¹, R³, R⁴, and R⁵ are hydrogen.

In other embodiments, R⁴ is C(O)R, wherein R is C₁₋₆alkyl. Exemplary C₁₋₆alkyl groups include methyl, ethyl, n-propyl, isopropyl, n-butyl, isobutyl, sec-butyl, and t-butyl, and isobutyl is often preferred.

In other embodiments, R³ is C(O)R, wherein R is C₁₋₆alkyl. Exemplary C₁₋₆alkyl groups include methyl, ethyl, n-propyl, isopropyl, n-butyl, isobutyl, sec-butyl, and t-butyl, and isobutyl is often preferred. In further embodiments, R³ can be C(O)R as defined above, R⁴ is hydrogen, and R² is a caffeic acid derivative. In preferred embodiments, the caffeic acid derivative is the compound wherein R^{b} and R^{c} are each hydroxyl, and R^{a}, R^{d}, and R^{e} are each hydrogen.

In some embodiments, R⁴ and R⁵ will together form a bond, yielding a lactone compound having the formula:

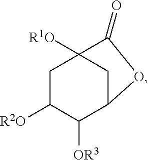

wherein R¹, R², and R³ have the meanings given above. In other embodiments, R⁵ and R² can form a bond, R⁵ and R³ can form a bond, or R⁵ and R¹ can form a bond.

In some instances, the flavor modulating compounds can include one or more of the following:

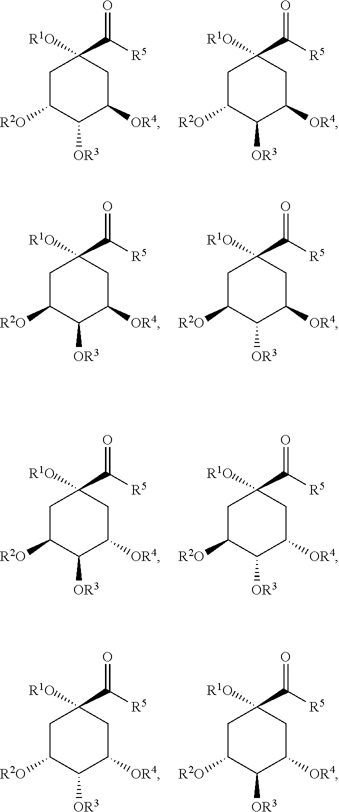

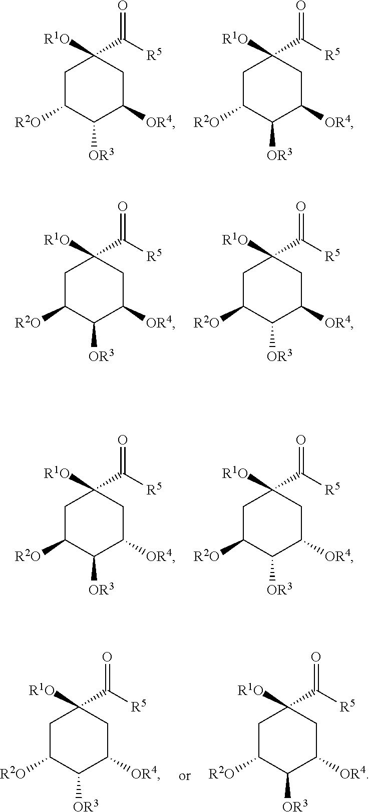

wherein R¹, R², R³, R⁴, and R⁵ are as defined above. The above compound may be present as a racemic mixture or an enantioenriched compounds, for instance, having an enantiomeric excess ee of at least 75%, 80%, 85%, 90%, 95%, 97.5%, 99%, or 99.5%. In preferred embodiments of the above compound, R³ is C(O)R, wherein R is C₁₋₆alkyl. Exemplary C₁₋₆₆alkyl groups include methyl, ethyl, n-propyl, isopropyl, n-butyl, isobutyl, sec-butyl, and t-butyl, and isobutyl is often preferred. In further embodiments, R³ can be C(O)R as defined above, R⁴ is hydrogen, and R² is a caffeic acid derivative. In preferred embodiments, the caffeic acid derivative is the compound wherein R^{b} and R^{c} are each hydroxyl, and R^{a}, R^{d}, and R^{e} are each hydrogen.

In certain embodiments, the flavor modulating compound can include one or more of the following:

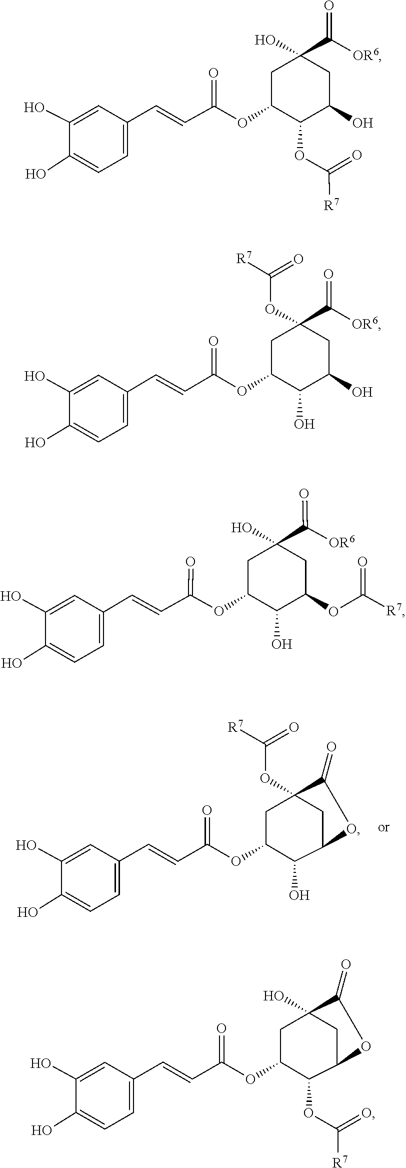

wherein R⁶ is selected from H, C₁₋₆alkyl, or benzyl; and R⁷ is selected from C₁₋₆alkyl, i.e., methyl, ethyl, n-propyl, isopropyl, n-butyl, sec-butyl, isobutyl, tert-butyl, etc. In cases where R⁶ is H, acceptable salts as defined above are also included.

The flavor modulating compounds disclosed herein may be obtained by selective esterification of chlorogenic acid, i.e, intramolecular lactonization, esterification of the 4-hydroxyl, followed by lactone hydrolysis. In other embodiments, the flavor modulating compounds may be obtained from coffee beans, preferably green coffee beans, most preferably green coffee beans that are considered below specialty grade. The flavor modulating compounds may be solvent extracted and purified using chromatographic methods.

The flavor modulating compounds may be used to reduce or eliminate the perception of bitter and/or sour tastes in a variety of foods and beverages. Moreover, the flavor modulating compounds can modulate the aroma and/or somatosensory characteristics of a food or beverage The compound may be added to a variety of different foods to increase palatability. For instance, the compounds may be added to vegetables, including cruciferous vegetables, yogurts, cranberries and other bitter fruits, cocoa, coffee, wine, or beer. In certain embodiments, the flavor modulating compounds can be used to mask the taste of anti-oxidants and preservatives, thereby increasing a food's shelf life without compromising its flavor. The flavor modulating compounds may be added to the wettable adhesives found in stamps and envelopes. The flavor modulating compounds may be added to medications, including liquid formulations, chewable formulations, dissolvable formulations, aerosol formulations, dry powder formulations, and spray formulations. By reducing bitterness and/or sourness, an increased adherence to a treatment regimen can be achieved, especially with pediatric patients. In other embodiments, the flavor modulating compounds may be combined with dental formulations, including topical anesthetics, adhesives, including denture adhesives, and cleaning products such as toothpastes, mouthwashes, and sealants.

The flavor modulating compounds may be added to foods and beverages in concentrations effective to block the bitter and/or sour tastes of the compounds contained therein. The compounds can augment or improve flavors as well as somatosensory effects (e.g., warming cooling sensations) in foods and beverages. The flavor modulating compounds can improve and/or augment the aromas associated with a particular food or beverage. For instance, the disclosed compounds may be added in an amount of at least 0.1 mg/kg, at least 0.5 mg/kg, at least 1 mg/kg, at least 2.5 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg, relative to the total weight of the consumable. In some embodiments, the flavor modulating compound can be added in an amount from 0.1-100 mg/kg, from 0.1-50 mg/kg, from 0.1-25 mg/kg, from 0.1-10 mg/kg, from 0.1-5 mg/kg, from 0.1-2.5 mg/kg, from 5-100 mg/kg, from 5-50 mg/kg, from 5-25 mg/kg, from 5-10 mg/kg, from 10-100 mg/kg, from 10-50 mg/kg, from 10-25 mg/kg, from 25-100 mg/kg, from 25-50 mg/kg, from 50-100 mg/kg, or from 75-100 mg/kg.

In certain embodiments, the flavor modulating compounds can be delivered to the oral cavity prior to consumption of the bitter product. The compounds can be formulated as a mouthwash, a lozenge, a lollipop, a chewable tablet, and the like. By pre-saturating the bitter and/or sour taste receptors in the tongue with the flavor modulating compounds, otherwise unpalatable substances may be more readily delivered to the oral cavity or consumed.

The compounds disclosed herein may be provided in an aqueous composition to more readily combine them with foods, beverages, and the like. The composition may be buffered, for instance at a pH between 6 and 8, between 6.5 and 8, between 6.5 and 7, between 6.5 and 7.5, between 6.5 and 8, between 7 and 8, or between 7.5 and 8. In other embodiments, the composition may be buffered at an acidic pH, for instance similar to found in citrus juice, vinegar, or yogurt. In some embodiment, the composition may be buffered at a pH between 2 and 8, between 2 and 7, between 2 and 6, between 2 and 5, between 2 and 4, between 3 and 8, between 3 and 7, between 3 and 6, between 3 and 5, between 3 and 4, between 4 and 8, between 4 and 7, between 4 and 6, between 4 and 5, between 5 and 8, between 5 and 7, or between 5 and 6.

The compounds may be provided in the composition at a concentration between about 0.1-100 mM, between about 0.5-100 mM, between about 1-100 mM, between about 5-100 mM, between about 10-100 mM, between about 25-100 mM, between about 50-100 mM, between about 0.1-50 mM, between about 0.1-25 mM, between about 0.1-10 mM, between about 0.1-5 mM, or between about 0.1-1 mM. When the aqueous composition contains more than one flavor modulating compound, the concentration refers to the total concentration of all the compounds.

### Example

5 panelists with extensive experience in sensory testing conducted a ‘difference from control’ test to evaluated the impact of compound 3-O-caffeoyl-4-O-3-methylbutanoylquinic acid (Compound 1) at a dosage level of 4 mg/l or 4 mg/kg (4 ppm) on the flavor profile to a wide range of food and beverage products. Panelists were asked to rate the degree of overall difference in the flavor profile between the sample (control) and the sample with added Compound 1 on a categorical scale of “no difference, slight different, moderate different, strong difference.”

Panelists also described any differences observed in the taste, aroma and somatosensory profile between the sample (control) and the sample with added Compound 1.

| | Noted Observed Differences in the Sample Flavor Profile with the |
|---|---|
| | Addition of 3-O-caffeoyl-4-O-3-methylbutanoylquinic acid at |
| Sample | dosage of 4 mg/L or 4 mg/Kg (Difference from Control Test) |
| 5% Cocoa in water | Moderate change in the cocoa flavor profile; characterized by |
| | modulation of the taste (sour, bitter), somatosensory (astringency) |
| | and aroma profile (burnt, smoky) profiles |
| Orange juice | Moderate change in the OJ flavor profile; characterized by |
| | modulation of the taste (sour) and aroma (green) profiles |
| Orange juice from | Strong change in the OJ flavor profile; characterized by modulation |
| huanglongbing (HLB) | of the taste (sour, bitter) and aroma profiles; enhancement of the |
| infected fruits from | orange juice character |
| Hamlin tree variety | |
| Orange juice from | Strong change in the OJ flavor profile; characterized by modulation |
| huanglongbing (HLB) | of the taste (sour, bitter) and aroma profiles; enhancement of the |
| infected fruits from | orange juice character |
| Valencia tree variety | |
| Non-alcoholic beer | Strong change in the beer flavor profile; characterized by |
| | modulation of the taste (sour) and somatosensory (irritation) |
| | profiles |
| Beer 3.2% | Moderate change in the beer flavor profile; characterized by |
| | modulation of somatosensory (metallic, itching, tingling, burn) |
| | profile |
| Beer 5.6% | Slight-moderate change in the beer flavor profile; characterized by |
| | modulation of the taste (sour) and somatosensory (metallic, |
| | tingling) profiles |
| Wine | Moderate change in the wine flavor profile; characterized by |
| | modulation of the taste (sour), somatosensory (astringency) and |
| | aroma (fruity, animalic) profiles |
| Stevia (400 mg/L) | Slight change in the stevia flavor profile; characterized by |
| | modulation of the taste (bitter) profile |
| Pea protein beverage | Slight change in the pea flavor profile; characterized by modulation |
| (11% protein) | of the somatosensory (mouthfeel) profile |
| Whole wheat bread | Moderate change in the bread flavor profile; characterized by |
| | modulation of the taste (sweet, bitter, salty, sour) and aroma (burnt, |
| | coffee) profiles |
| Nonfat Greek yogurt | Strong change in the yogurt flavor profile; characterized by |
| | modulation of the taste (sour), somatosensory (astringency, |
| | mouthfeel) and aroma (buttery) profiles |
| Whisky | Strong change in the whisky flavor profile; characterized by |
| | modulation of the taste (sour, sweetness) and somatosensory (burn, |
| | irritation) profiles |
| Tequila | Slight change in the tequila flavor profile; characterized by |
| | modulation of the somatosensory (burn, irritation) and aroma |
| | (peppery, fruity) profiles |

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A method for modulating the flavor in a composition, comprising adding to the composition at least one compound having the formula: 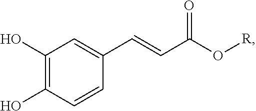
or an acceptable salt thereof,
wherein R is C₁₋₈alkyl, C₂₋₈alkenyl, C₂₋₈alkynyl, aryl, C₁₋₈heteroaryl, C₃₋₈cycloalkyl, or C₁₋₈heterocyclyl.

2. The method according to claim 1, wherein R comprises a C₃₋₈cycloalkyl or aryl group having the formula: 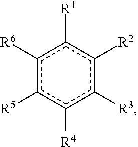
wherein one or more of R¹, R², R³, R⁴, R⁵, or R⁶ represents a bond to a cinnamoyl group, [symbol] independently represents a single or double bond, as permitted by valence, and the remaining five groups are selected from hydrogen, OH, C(O)OH, O—C₁₋₈alkyl, C(O)O—C₁₋₈alkyl, C₁₋₈alkyl, CH₂OH, and CH₂OC₁₋₈alkyl.

3. The method according to claim 1, wherein the compound has the formula: 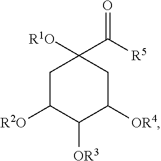
or an acceptable salt thereof,
wherein one of R¹, R², R³, or R⁴ is a caffeic acid derivative having the formula: 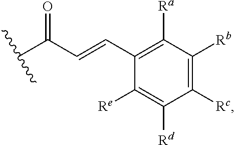
wherein R^{a}, R^{b}, R^{c}, R^{d}, and R^{e} are independently selected from F, Cl, Br, I, nitro, R, OR, N(R)₂, SO₂R, SO₂N(R)₂, C(O)R; C(O)OR, OC(O)R; C(O)N(R)₂, N(R)C(O)R, OC(O)N(R^{1b′})₂, N(R)C(O)N(R)₂, wherein R is in each case independently selected from hydrogen, C₁₋₆alkyl, C₂₋₈ alkenyl, C₂₋₈alkynyl, aryl, C₁₋₈heteroaryl, C₃₋₈cycloalkyl, or C₁₋₈heterocyclyl;
the remaining R¹, R², R³, and R⁴ are independently selected from hydrogen, C(O)R; C(O)OR, and C(O)N(R)₂, wherein R is in each case independently selected from hydrogen, C₁₋₈ alkyl, C₂₋₈alkenyl, C₂₋₈alkynyl, aryl, C₁₋₈heteroaryl, C₃₋₈cycloalkyl, or C₁₋₈heterocyclyl; and
R⁵ is selected from OR or NR₂, wherein R is independently selected from hydrogen, C₁₋₈ alkyl, C₂₋₈alkenyl, C₂₋₈alkynyl, aryl, C₁₋₈heteroaryl, C₃₋₈cycloalkyl, or C₁₋₈heterocyclyl, wherein R⁵ may form a bond with any of R¹, R², R³, or R⁴.

4. The method of claim 3, wherein the compound has the formula:

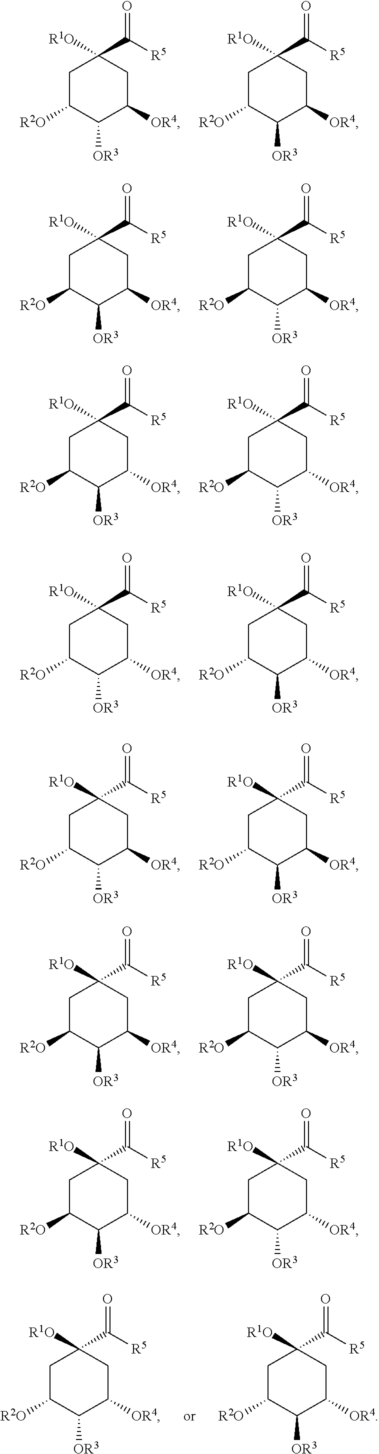

5. The method of claim 1, wherein the compound comprises one or more of the following: 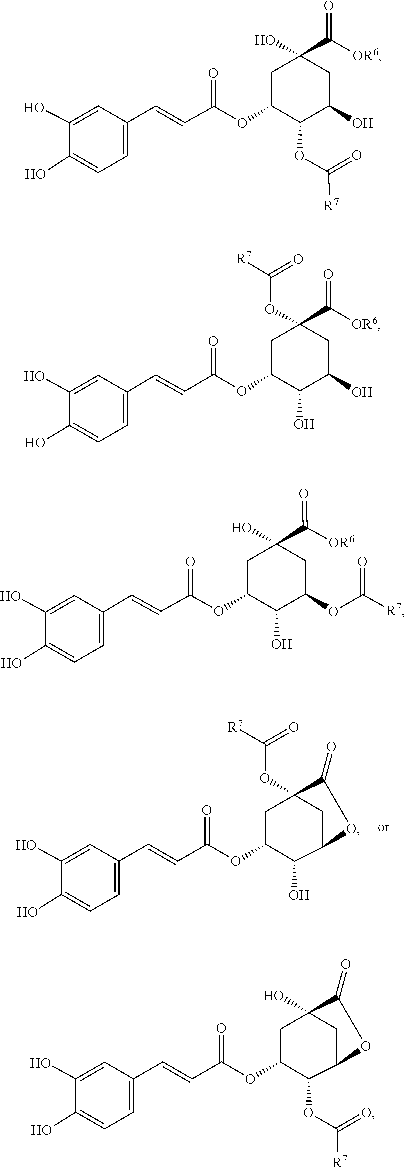
wherein R⁶ is selected from H, C₁₋₆alkyl, or benzyl; and R⁷ is selected from C₁₋₆alkyl.

6. The method according to claim 1, wherein the composition comprises a food, beverage, medication, or adhesive.

7. The method according to claim 1, wherein the composition comprises orange juice, coffee, beer, wine, or a distilled spirit.

8. The method according to claim 1, wherein the composition comprises bread, yogurt, vegetables, or fruit.

9. A composition comprising water, buffered to a pH between 2 and 8, and at least one compound having the formula: 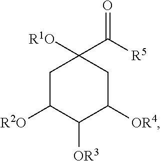
or an acceptable salt thereof,
wherein at least one of R¹, R², R³, or R⁴ is a caffeic acid derivative having the formula: 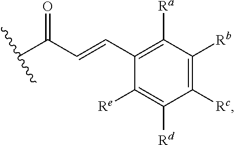
wherein R^{a}, R^{b}, R^{c}, R^{d}, and R^{e} are independently selected from F, Cl, Br, I, nitro, R, OR, N(R)₂, SO₂R, SO₂N(R)₂, C(O)R; C(O)OR, OC(O)R; C(O)N(R)₂, N(R)C(O)R, OC(O)N(R^{1b′})₂, N(R)C(O)N(R)₂, wherein R is in each case independently selected from hydrogen, C₁₋₈alkyl, C₂₋₈ alkenyl, C₂₋₈alkynyl, aryl, C₁₋₈heteroaryl, C₃₋₈cycloalkyl, or C₁₋₈heterocyclyl;
the remaining R¹, R², R³, and R⁴ are independently selected from hydrogen, C(O)R; C(O)OR, and C(O)N(R)₂, wherein R is in each case independently selected from hydrogen, C₁₋₈ alkyl, C₂₋₈alkenyl, C₂₋₈alkynyl, aryl, C₁₋₈heteroaryl, C₃₋₈cycloalkyl, or C₁₋₈heterocyclyl; and
R⁵ is selected from OR or NR₂, wherein R is independently selected from hydrogen, C₁₋₈ alkyl, C₂₋₈alkenyl, C₂₋₈alkynyl, aryl, C₁₋₈heteroaryl, C₃₋₈cycloalkyl, or C₁₋₈heterocyclyl, wherein R⁵ may form a bond with any of R¹, R², R³, or R⁴.

10. The composition of claim 9, wherein the compound has the formula:

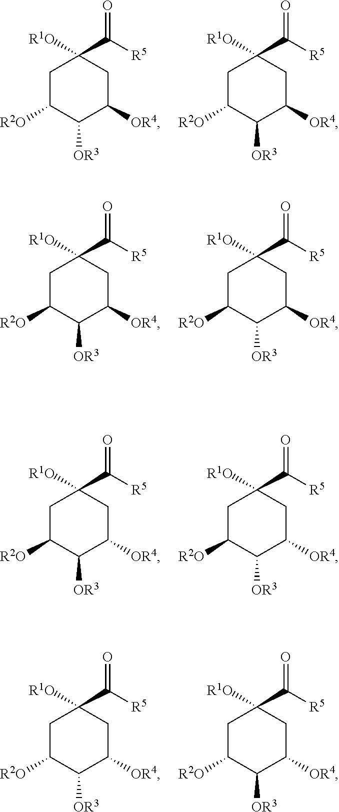

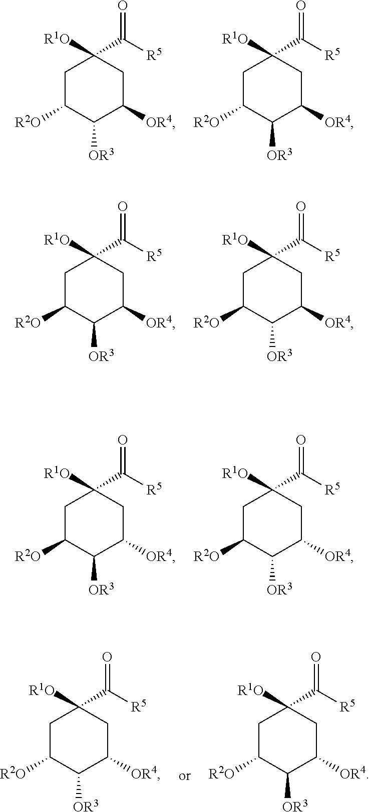

11. The composition of claim 9, wherein the compound comprises one or more of the following: 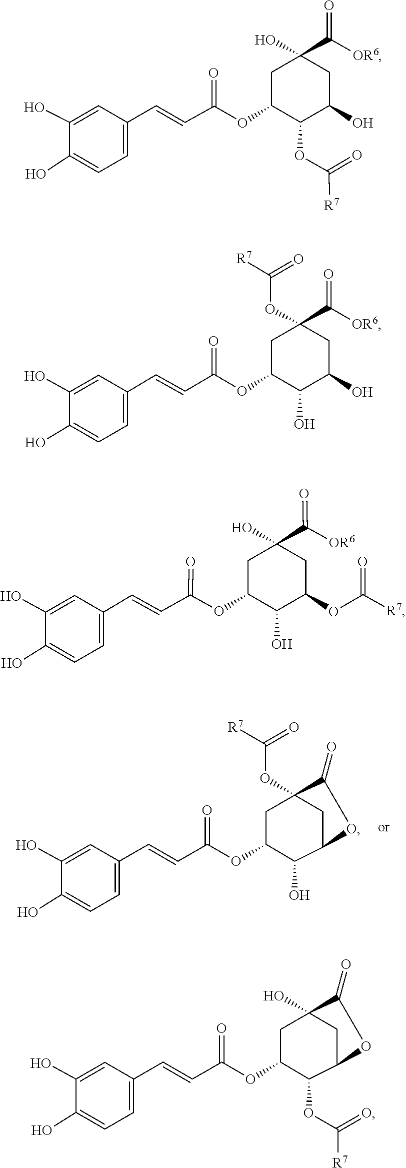
wherein R⁶ is selected from H, C₁₋₆alkyl, or benzyl; and R⁷ is selected from C₁₋₆alkyl.

12. The composition according to claim 9, wherein the compound is present at a concentration between about 0.1-100 mM.

13. The composition according to claim 9, wherein the water is buffered to a pH between 3 and 6 or 6 and 8.
